(19) United States
(12) Patent Application Publication
(10) Pub. No.: US 2022/0041544 A1
(43) Pub. Date: Feb. 10, 2022

(54) AZOBENZENE DERIVATIVE, PREPARATION METHOD THEREOF, AZOPHENYL LIGHT CONTROL REVERSIBLE ADHESIVE AND METHOD OF USING THE SAME

(71) Applicant: SHENZHEN CHINA STAR OPTOELECTRONICS SEMICONDUCTORS DISPLAY TECHNOLOGY CO., LTD., Shenzhen, Guangdong (CN)

(72) Inventor: Haoxu WU, Shenzhen (CN)

(73) Assignee: Shenzhen China Star Optoelectronics Semiconductor Display Technology Co., Ltd., Shenzhen, Guangdong (CN)

(21) Appl. No.: 16/980,447

(22) PCT Filed: Sep. 7, 2020
(86) PCT No.: PCT/CN2020/113756

§ 371 Date: Sep. 14, 2020

(30) Foreign Application Priority Data
Aug. 7, 2020 (CN) 202010787570.5

(51) Int. Cl. C07C 245/08; C09J 11/06; C09J 5/00; H01L 51/00; B32B 37/12; B32B 43/00

(52) U.S. Cl. CPC C07C 245/08; C09J 11/06; C09J 5/00; H01L 51/003; H01L 51/0097; G02F 1/133305; B32B 43/006; C09J 2203/326; H01L 2227/326; H01L 2251/5338; G02F 2202/28; B32B 37/1284

(57) ABSTRACT

An azobenzene derivative, a preparation method of the azobenzene derivative, an azophenyl light control reversible adhesive, and a method of using the azophenyl light control reversible adhesive are disclosed. The azobenzene derivative has a molecular structure as P1 or P2. In the abovementioned preparation method, the azobenzene derivative P1 or P2 is obtained from an esterification reaction of an azophenyl group, 3,4,5-tripentyloxybenzoic acid or 3,4,5-tri(dodecyloxy)benzoic acid, and 2,2′-dihydroxy-1,1′-binaphthol. The melting point of the azobenzene derivative P1 or P2 is modified commonly by alkyl chains and binaphthol to be slightly higher than room temperature.

### FIELD OF INVENTION

The present disclosure belongs to a technical field of adhesive materials, and specifically relates to an azobenzene derivative, a preparation method thereof, an azophenyl light control reversible adhesive, and a method of using the same.

### BACKGROUND OF INVENTION

Flexible displays have the characteristics of lightness and thinness, rollability, impact resistance, etc., and have a huge potential market in the field of portable electronic products. A liquid crystal display (referred to as LCD) and an organic light emitting display (referred to as OLCD) both can achieve flexible displays.

In the process of preparing flexible displays, a key technology for preparing flexible devices in the field of flexible display technology is how to separate the flexible substrate from the carrier substrate. In the prior art, mechanical lift-off or laser lift-off technology is usually used. The laser lift-off technology causes less damage to the flexible substrate, so it is widely used. In the laser lift-off technology, the flexible substrate is disposed on the glass substrate (that is, the glass substrate is used as the carrier substrate), and then the display components are prepared on the flexible substrate. After the preparation, the contact interface between the glass substrate and the flexible substrate is irradiated with a laser so as to separate the flexible substrate from the glass substrate.

### Technical Problem

When the laser irradiates the contact interface between the glass substrate and the flexible substrate, the energy may also be absorbed by the flexible substrate, causing the flexible substrate to ablate and release gas. Therefore, the flexible substrate expands partly and forms wrinkles after cooling, and even local stress is excessive and the flexible substrate is warped, causing product defects.

### SUMMARY OF DISCLOSURE

### Technical Solutions

For solving the above technical problems, the present disclosure provides an azobenzene derivative, a preparation method thereof, an azophenyl light control reversible adhesive and a method of using the same.

In order to solve above technical problems, the present disclosure provides a technical solution which is:

an azobenzene derivative, having a molecular structure shown as below:

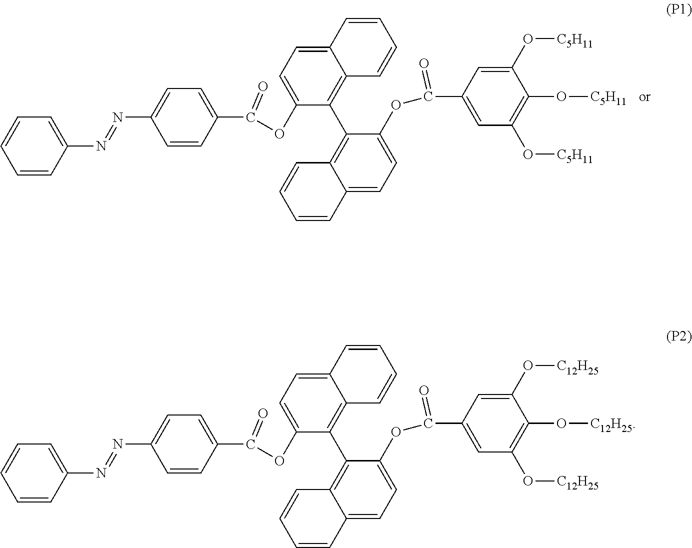

An azophenyl light control reversible adhesive, comprising abovementioned azobenzene derivative P1 and/or P2.

A method of using the abovementioned azophenyl light control reversible adhesive, comprising steps of:

SS1: coating an azophenyl light control reversible adhesive in a liquid state on a surface of a flexible substrate opposite to a glass substrate, so as to adhere the flexible substrate to the glass substrate, and then standing for a predetermined time;

SS2: forming display components on the flexible substrate;

SS3: irradiating the glass substrate with green light; and

SS2: applying opposite forces to the flexible substrate and the glass substrate, respectively, to separate the flexible substrate and the glass substrate.

### Beneficial Effect

In the present disclosure, the melting point of an azobenzene derivative P1 or P2 is modified commonly by alkyl chains and binaphthol to be slightly higher than room temperature. P1 and P2 in a liquid state may be cured spontaneously and quickly within 2 minutes and then exhibit an excellent bonding property after cured. The bonding strength can reach several megapascals to meet the requirements of subsequent processes. Under the irradiation of green light, the photothermal effect of the azobenzene derivative P1 or P2 causes the conversion from solid to liquid. After the irradiation of green light (under the same conditions of power and time, only green light (500-550 nm, 175 mW cm-2, 480 s-500 s) can achieve the conversion from solid to liquid of the azobenzene derivative P1 or P2), the azobenzene derivative P1 and P2 are heated to be melted and lose their bonding properties. This process is highly reversible. The heat in the process can be isolated on one side of the outer glass by the porous structure composed of ceramic fiber particles added. While heat cannot penetrate to one side of the panel, the middle layer peels off. This optical switch adhesive (azophenyl light control reversible adhesive) is easily soluble in common organic solvents, such as methylene chloride, easily cleaned and recovered, and can be recycled. The method is simple to operate and has low costs. The method may eliminate product defects caused by laser stripping, and can be used as a potential technology for stripping a flexible substrate.

### BRIEF DESCRIPTION OF DRAWINGS

FIG. 1 shows images of temperature and solid-liquid transition of an azobenzene derivative P1 with the irradiating time of green light in the present disclosure.

FIG. 2 shows solid absorption spectra of azobenzene derivatives P1 and P2 of the present disclosure.

FIG. 3 is a schematic view showing the working of an azophenyl light control reversible adhesive according to the present disclosure.

### DETAILED DESCRIPTION OF PREFERRED EMBODIMENTS

In order to make the technical problems to be solved by the present disclosure, technical solutions, and beneficial effects clearer, the following further describes the present disclosure in detail with reference to the accompanying drawings and embodiments. It should be understood that the specific embodiments described here are merely to explain the present disclosure, but not to limit the present disclosure.

In the present disclosure, the melting point of an azobenzene derivative P1 or P2 is modified commonly by alkyl chains and binaphthol to be slightly higher than room temperature. P1 and P2 in a liquid state may be cured spontaneously and quickly within 2 minutes and then exhibit an excellent bonding property after cured. The bonding strength can reach several megapascals to meet the requirements of subsequent processes. Under the irradiation of green light, the photothermal effect of the azobenzene derivative P1 or P2 causes the conversion from solid to liquid. After the irradiation of green light (under the same conditions of power and time, only green light (500-550 nm, 175 mW cm-2, 480 s-500 s) can achieve the conversion from solid to liquid of the azobenzene derivative P1 or P2), the azobenzene derivative P1 and P2 are heated to be melted and lose their bonding properties. This process is highly reversible. The heat in the process can be isolated on one side of the outer glass by the porous structure composed of ceramic fiber particles added. While heat cannot penetrate to one side of the panel, the middle layer peels off. This optical switch adhesive (azophenyl light control reversible adhesive) is easily soluble in common organic solvents such as methylene chloride, easy to clean and recover, and can be recycled. The method is simple to operate and has low cost. The method may eliminate product defects caused by laser stripping, and can be used as a potential technology for stripping a flexible substrate.

### Embodiment 1

An azobenzene derivative, denoted as P1, having a molecular structure as below:

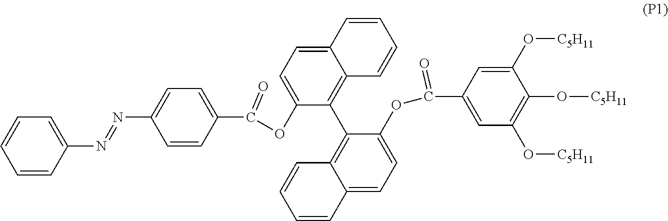

A preparation method of the azobenzene derivative P1 is as below: azobenzene, 3,4,5-tripentyloxybenzoic acid, 2,2′-dihydroxy-1,1′-binaphthol are provided with an amount ratio of 1:1:1; the above three raw materials are added into a reactor containing water to react together; the reactants are evenly stirred at 20° C. to react for 30 mins; the reaction mixture is then filtered (suction filtration) and dried; and an orange solid obtained is the azobenzene derivative P1. The melting point measured is 44° C.

The preparation method of the present disclosure is simple and low-cost. The reaction can take place at room temperature with water as the medium without catalyst, and the conversion rate of raw materials is as high as 85%-93%.

P1, as the optical switch adhesive (azophenyl light control reversible adhesive), is easily soluble in common organic solvents such as methylene chloride, easily cleaned and recovered, and can be recycled.

### Embodiment 2

The preparation method of the azobenzene derivative P1 in this embodiment is as below: azobenzene, 3,4,5-tripentyloxybenzoic acid, 2,2′-dihydroxy-1,1′-binaphthol are provided with an amount ratio of 1:0.5:1.5; the above three raw materials are added into a reactor containing water to react together; the reactants are evenly stirred at 25° C. to react for 20 mins; the reaction mixture is then filtered (suction filtration) and dried; and an orange solid obtained is the azobenzene derivative P1.

### Embodiment 3

The preparation method of the azobenzene derivative P1 in this embodiment is as below: azobenzene, 3,4,5-tripentyloxybenzoic acid, 2,2′-dihydroxy-1,1′-binaphthol are provided with an amount ratio of 1:1.5:0.5; the above three raw materials are added into a reactor containing water to react together; the reactants are evenly stirred at 30° C. to react for 25 mins; the reaction mixture is then filtered (suction filtration) and dried; and an orange solid obtained is the azobenzene derivative P1.

### Embodiment 4

An azobenzene derivative, denoted as P2, having a molecular structure as below:

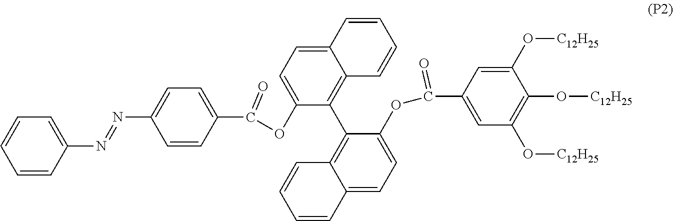

The preparation method of the azobenzene derivative P1 is as below: azobenzene, 3,4,5-tri(dodecyloxy)benzoic acid, 2,2′-dihydroxy-1,1′-binaphthol are provided with an amount ratio of 1:1:1; the above three raw materials are added into a reactor containing water to react together; the reactants are evenly stirred at 20° C. to react for 20 mins; the reaction mixture is then filtered (suction filtration) and dried; and an orange solid obtained is the azobenzene derivative P2.

### Embodiment 5

The preparation method of the azobenzene derivative P1 in this embodiment is as below: azobenzene, 3,4,5-tri(dodecyloxy)benzoic acid, 2,2′-dihydroxy-1,1′-binaphthol are provided with an amount ratio of 1:0.5:1.5; the above three raw materials are added into a reactor containing water to react together; the reactants are evenly stirred at 25° C. to react for 30 mins; the reaction mixture is then filtered (suction filtration) and dried; and an orange solid obtained is the azobenzene derivative P2.

### Embodiment 6

The preparation method of the azobenzene derivative P1 in this embodiment is as below: azobenzene, 3,4,5-tri(dodecyloxy)benzoic acid, 2,2′-dihydroxy-1,1′-binaphthol are provided with an amount ratio of 1:1.5:0.5; the above three raw materials are added into a reactor containing water to react together; the reactants are evenly stirred at 30° C. to react for 25 mins; the reaction mixture is then filtered (suction filtration) and dried; and an orange solid obtained is the azobenzene derivative P2

### Embodiment 7

An azophenyl light control reversible adhesive comprises the azobenzene derivative P1 and/or P2 in any of Embodiments 1-6.

A method of using the azophenyl light control reversible adhesive comprises below steps of:

SS1: coating an azophenyl light control reversible adhesive in a liquid state on a surface of a flexible substrate opposite to a glass substrate, so as to adhere the flexible substrate to the glass substrate, and then standing for a predetermined time greater than or equal to 2 minutes, for example, 3 minutes, so as to make the azophenyl light control reversible adhesive to be cured quickly under room temperature;

SS2: forming display components on the flexible substrate;

SS3: irradiating the glass substrate with green light, wherein a time of irradiating with the green light is greater than or equal to 8 minutes, for example, 9 minutes, so as to allow the azophenyl light control reversible adhesive to convert from solid to liquid; and

SS2: applying opposite forces to the flexible substrate and the glass substrate, respectively, to separate the flexible substrate and the glass substrate.

The method is simple and low-cost. The azophenyl light control reversible adhesive is easily soluble in common organic solvents such as methylene chloride, easily cleaned and recovered, and can be recycled. It may eliminate product defects caused by laser stripping, and can be used as a potential technology for stripping a flexible substrate.

### Test Example 1

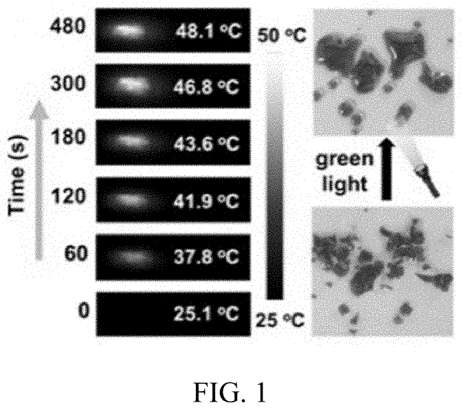

The azobenzene derivative P1 obtained in Embodiment 1 is placed at room temperature and irradiated with green light (500-550 nm, 175 mW cm-2) for 480 seconds. The state and temperature changes of the azobenzene derivative P1 are recorded. As shown in FIG. 1, after the green light is continuously irradiated for 480 s, the temperature of the azobenzene derivative P1 rises to 50° C., and the azobenzene derivative P1 changes from a solid to a liquid. It shows that, under the irradiation of green light, the photothermal effect of P1 does cause the conversion from a solid to a liquid, and the phenomenon of solid-liquid conversion is very significant.

### Test Example 2

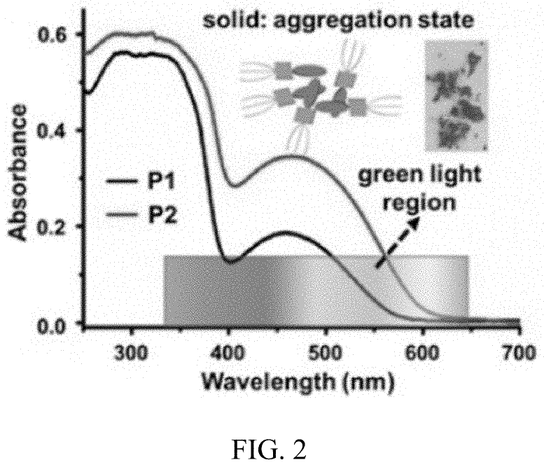

The absorption spectra of 5 mg of azobenzene derivative P1 obtained in Embodiment 1 and 5 mg of azobenzene derivative P2 obtained in Embodiment 4 are measured to proof that the azobenzene derivative P1 and the azobenzene derivative P2 do have absorption within green light area as shown in FIG. 2.

### Test Example 3

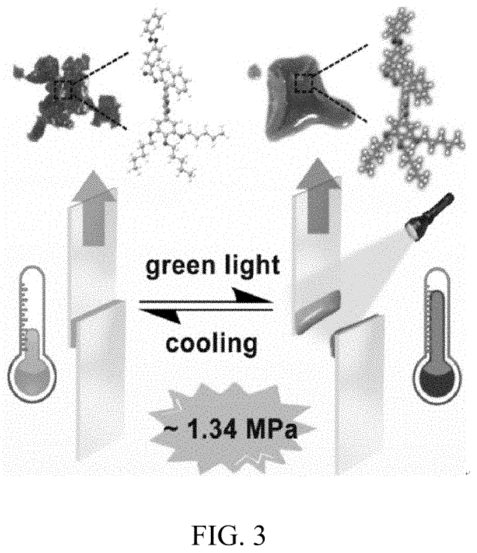

The azobenzene derivative P1 is coated on a surface of a flexible substrate opposite to a glass substrate, so as to adhere the flexible substrate to the glass substrate, and then stands for 2 minutes to cure. The cured P1 has a bonding strength reaching to 1.34 MPa as shown in FIG. 3.

After irradiated with green light for 500 seconds, the azobenzene derivative P1 converts to liquid state and basically loses the bonding effect. When the irradiation is stopped, along with the release of heat, the liquid azobenzene derivative P1 quickly and spontaneously returns to the solid state within 2 minutes, and the bonding strength reaches to 1.32 MPa after the azobenzene derivative P1 is cured.

In the present disclosure, the melting point of an azobenzene derivative P1 or P2 is modified commonly by alkyl chains and binaphthol to be slightly higher than room temperature (44° C. and 53° C., respectively). P1 and P2 in liquid state may be cured spontaneously and quickly within 2 minutes and then exhibit an excellent bonding property after cured. The bonding strength can reach several megapascals to meet the requirements of subsequent processes. Under the irradiation of green light, the photothermal effect of the azobenzene derivative P1 or P2 causes the conversion from solid to liquid. After the irradiation of green light, the azobenzene derivative P1 and P2 are heated to be melted and lose their bonding properties. This process is highly reversible. The heat in the process can be isolated on one side of the outer glass by the porous structure composed of ceramic fiber particles added. While heat cannot penetrate to one side of the panel, the middle layer peels off.

The above descriptions are merely preferred embodiments of the present disclosure. For those of ordinary skill in the art, based on the idea of the present disclosure, many changes may be made in specific implementations and applications. These changes all belong to the protection scope of the present disclosure as long as these changes do not depart from the concept of the present disclosure.

What is claimed is:

1. An azobenzene derivative, having a molecular structure shown as below:

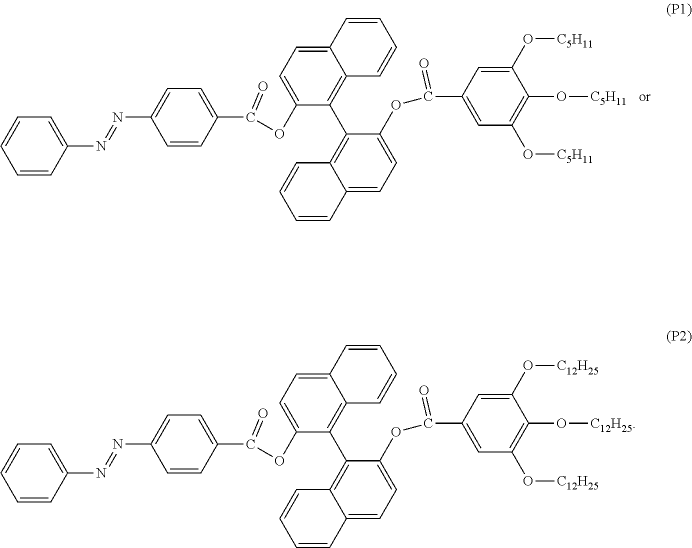

2. A preparation method of an azobenzene derivative according to claim 1, wherein an azobenzene derivative P1 or P2 is obtained from an esterification reaction of an azophenyl group, 3,4,5-tripentyloxybenzoic acid or 3,4,5-tri(dodecyloxy)benzoic acid, and 2,2′-dihydroxy-1,1′-binaphthol.

3. The preparation method of the azobenzene derivative according to claim 2, wherein a reaction medium for the esterification reaction of an azophenyl group, 3,4,5-tripentyloxybenzoic acid or 3,4,5-tri(dodecyloxy)benzoic acid, and 2,2′-dihydroxy-1,1′-binaphthol is water, and adding the above three raw materials into water to react together, so as to obtain the azobenzene derivative P1 or P2.

4. The preparation method of the azobenzene derivative according to claim 2, wherein a reaction time is 20-40 minutes.

5. The preparation method of the azobenzene derivative according to claim 2, wherein a reaction temperature is 20-30° C.

6. The preparation method of the azobenzene derivative according to claim 2, wherein an amount ratio of the azophenyl group to 3,4,5-tripentyloxybenzoic acid or 3,4,5-tri(dodecyloxy)benzoic acid to 2,2′-dihydroxy-1,1′-binaphthol is 1:(0.5-2):(0.5-2).

7. An azophenyl light control reversible adhesive, comprising the azobenzene derivative P1 and/or P2 according to claim 1.

8. A method of using an azophenyl light control reversible adhesive, comprising steps of:
SS1: coating an azophenyl light control reversible adhesive in a liquid state on a surface of a flexible substrate opposite to a glass substrate, so as to adhere the flexible substrate to the glass substrate, and then standing for a predetermined time;
SS2: forming display components on the flexible substrate;
SS3: irradiating the glass substrate with green light; and
SS2: applying opposite forces to the flexible substrate and the glass substrate, respectively, to separate the flexible substrate and the glass substrate.

9. The method of using the azophenyl light control reversible adhesive according to claim 8, wherein the predetermined time of standing in the step SS1 is greater than or equal to 2 minutes.

10. The method of using the azophenyl light control reversible adhesive according to claim 8, wherein a time of irradiating with the green light in the step SS3 is greater than or equal to 8 minutes.
